(19) United States
(12) Patent Application Publication
(10) Pub. No.: US 2024/0115348 A1
(43) Pub. Date: Apr. 11, 2024

(54) Device for Performing Sterile Invasive Medical Procedures That Facilitates Ultrasound Guidance During Procedures

(71) Applicant: Donald Robert Wallace, Hanna City, IL (US)

(72) Inventor: Donald Robert Wallace, Hanna City, IL (US)

(21) Appl. No.: 17/803,672

(22) Filed: Oct. 11, 2022

(51) Int. Cl. A61B 90/40; A61G 10/00

(52) U.S. Cl. CPC A61B 90/40; A61G 10/005; A61B 2034/2063

(57) ABSTRACT

A system for use in invasive medical procedures of the skin with a plastic clear tent like structure that is internally sterile and has incorporated gloves, attaches to the skin, and allows any devices contained in the system to be manipulated in sterile fashion to accomplish a number of different medical procedures while facilitating ultrasound guidance of procedures.

### CROSS REFERENCE

63/360,526:

Filed: Oct. 12, 2021

Device for Performing Invasive Medical Procedures to Facilitate Rapid Achievement of Sterile Field and Separate External and Internal Work Areas Manipulated Through the Device Which would Also Facilitate Ultrasound Guidance.

### DESCRIPTION

Field of invention. The invention relates to the field of medicine and the performance of various invasive procedures where the devices used and procedural areas that contact skin and other body surfaces are maintained in a sterile condition to prevent bacterial contamination during said procedure.

### BACKGROUND OF INVENTION

Invasive medical procedures are commonly performed in the medical fields. These procedures include but are not limited to intravenous line placement, central venous line placement, arterial line placement, chest tubes, and lumbar puncture wherein a catheter or other percutaneous device is placed into a vessel or other structure through the skin under sterile conditions. A typical placement of such a device requires establishment of a sterile field to prevent bacterial infection of skin or blood from required penetration of skin during the procedure. The skin is generally treated with sterilizing agents such as chlorhexidine or iodine. The operator will undergo a preprocedural routine that usually includes donning of cap, mask then a sterile gown and gloves which consume a great deal of time. The skin is then covered with sterile drapes made of cloth, paper, or plastic with an opening provided over the procedural skin site where skin puncture will occur. The sterile devices needed to perform the procedure are uncovered from their packaging and kept within their containers nearby or placed on to the drape using sterile technique. The procedure is then performed often with skin puncture after the above. In the case of venous or arterial catheterization the procedure will often require the use of guidewires through a needle after syringe aspiration of blood to facilitate catheter placement and devices to secure the catheter or other device placed during the procedure. Often ultrasound will be used to facilitate a procedure and guide a needle or other device to be placed by locating a suitable vessel or other structure and the ultrasound then guides the placement of the needle or other device into the vessel or other structure. The ultrasound probe is necessarily placed in close proximity to the puncture site to visualize structures that will be manipulated and to avoid some adjacent structures so the probe should be sterile or have a sterile barrier between the probe and skin. There are a number of problematic issues with the typical invasive medical procedure as described above:
1) The establishment of the sterile field with donning of sterile attire and placement of sterile drapes usually consumes a great deal of time and material.
2) The sterile field is at times extended over the patients face and upper and lower body obscuring ability to monitor respiratory status and other important vital patient status information.
3) The sterile field may be easily contaminated by accidental and even unnoticed intrusion by unsterile entities such a contaminated sterile gloved hand or component that was contaminated and transferred to the the sterile field. The typical sterile field is also open to circulating ambient air and thus airborne contaminants are a concern. Some hospitals use dedicated observers to monitor for breach of sterile protocol during procedures as that breach occurs frequently and is sometimes unnoticed.
4) Ultrasound assisted placement of devices involving skin puncture usually requires an unsterile ultrasound probe to be covered with a sterile probe cover by the proceduralist as it will be used in close proximity to the procedure and contacts the sterile area. This is time consuming, usually requires an assistant and is another opportunity to contaminate the sterile field as the sterile hand of the proceduralist will be in close proximity to the unsterile probe while the probe cover is placed.
It is important to identify causes of infection related to performing invasive procedures. There are many steps involved in sterile preparation of the operator and area for procedure and thus many steps where bacterial contamination can be introduced. Furthermore the ambient air circulating over the area of the procedure is known to contain bacteria that can cause contamination of the sterile field. It is important to minimize the likelihood of contamination and the invention does so while also offering convenience of time and material savings as well as improved patient monitoring during the procedure.

### SUMMARY OF THE INVENTION

The present invention seeks to remedy the above limitations of typical invasive procedure performance utilizing a device that incorporates the sterile procedural components in an internally sterile device with incorporated gloves that allows for the device containing the components to serve as an effective internal sterile working area for manipulation of the contained components and will also allow sterile use of an ultrasound probe as needed in the area adjacent to skin puncture to facilitate the procedure. The use of the device results in reduced time needs, reduced likelihood of infections, better monitoring of the patient during procedure as reduced sterile drape coverage of the patient is required, reduced material requirements, and facilitates ultrasound guidance compared to traditional sterile preparation.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 is a top view of the invention.

FIG. 2 is a side view of the invention.

FIG. 3 is an exploded side view of the opening in the device for attachment to the skin.

FIG. 4 is an exploded side view of a resealable flap on the device.

### DESCRIPTION OF THE INVENTION

The current and generally accepted method of performing sterile percutaneous procedures requires sterilization of the skin surface where the procedure is to be performed with cleaning agents such as chlorhexidine or iodine. The person performing the procedure will don a cap, mask and sterile gown and gloves in a sterile fashion before covering the working and surrounding areas of the skin with a large sterile drape that typically covers large areas of the patient. The sterile drape usually has an opening for skin puncture. The process of achieving the sterile working conditions is time consuming and fraught with opportunities for error involving bacterial contamination during the steps to accomplish this including airborne particles that come to rest on sterile field from ambient air, proceduralist and assistants errors and multiple opportunities for contamination during donning of gown and gloves and during the procedure itself. During the procedure for example a guidewire may contact a non sterile area without being noticed before it is placed into a needle that has cannulated a blood vessel or the proceduralist's sterile hand may contact a non sterile area surrounding the sterile field. A large quantity of material is also consumed for the draping as well and it often covers the patient's face making it difficult to monitor patient status during the procedure.

Ultrasound is often utilized in these procedures to facilitate placement of needles and other devices into structures such as blood vessels or fluid collections. An ultrasound probe is placed over an area to identify structures, often blood vessels or fluid collections, that are to be accessed and often cannulated or drained during the procedure. The ultrasound probe can be used to visualize needles or other instruments in real time as they enter a blood vessel or other structure during the procedure. In order to keep the unsterile probe from direct contact with the sterilized skin and thus causing contamination it is usually covered with a sterile probe cover that is placed partially by the proceduralist who the extends the rest of the cover to an assistant who pulls the remaining telescoping portion of the cover towards the cable connected to the probe thus covering most of the cable as it extends toward the ultrasound machine. This is time consuming and the transfer of the probe cover involves close proximity of the proceduralist and the assistant who is usually not in sterile attire thus providing another opportunity for contamination. The unsterile ultrasound probe is in close proximity to the sterile hand of the proceduralist during the placement of the probe cover and thus a source of possible contamination.

The invention will remedy the concerns of time and material consumption as well as problems related to bacterial contamination by providing a transparent self contained sterile tent-like internal area with incorporated gloved projections to perform the procedure within a structure that allows manipulation of contained instruments and visibility needed to perform the procedure in a rapid and sterile manner. The invention will also remedy the pitfalls of ultrasound probe use by providing a tubular pocket which the probe can be placed in to perform ultrasound guidance without need for covering the probe separately and an unsterile hand of the proceduralist or assistant can be used to place the probe into the pocket.

The dimensions of the invention will vary depending on, the body area of the procedure, the size and number of devices utilized and the requirements of the procedure involved

The invention could be used as a stand alone internally sterile procedural container available in different sizes and sterile components needed for the procedure added through the resealable opening just prior to the procedure before or after skin attachment of the device. Alternatively and optimally procedural devices and materials needed to perform procedures, such as but not limited to, central lines could be placed in the invention when manufactured and sterilized at the place of manufacture.

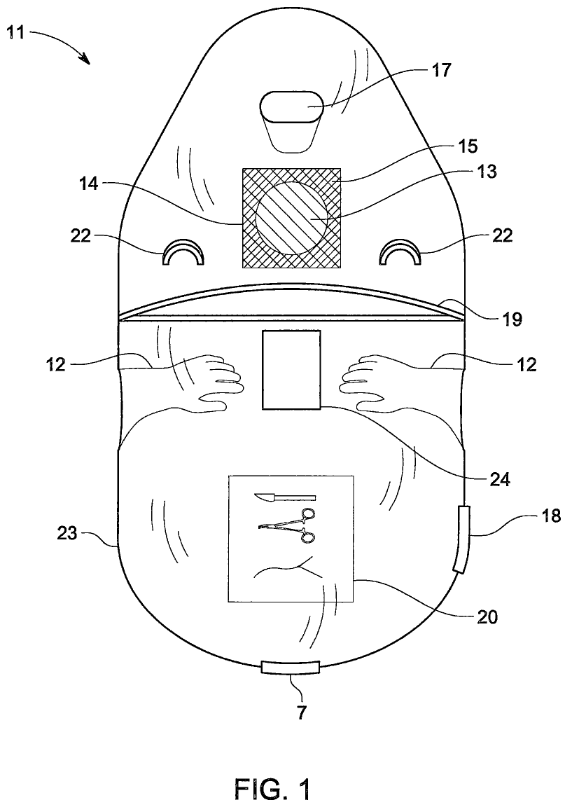

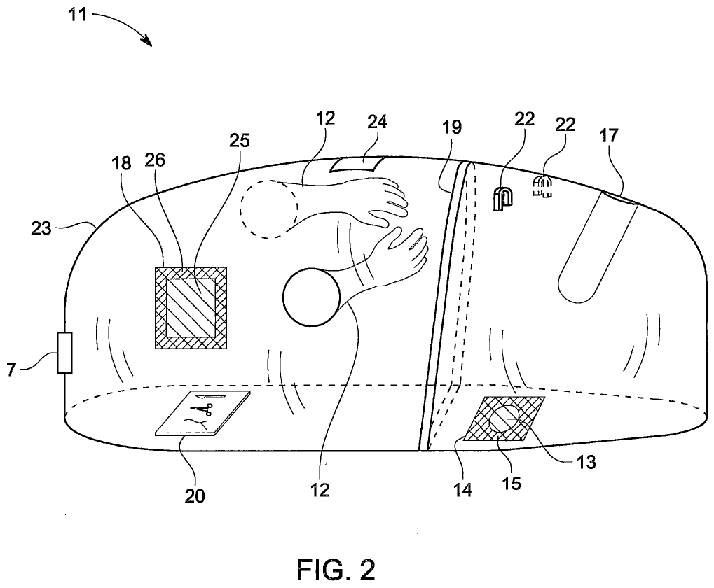

FIGS. 1-2 demonstrate a device for performing invasive medical procedures 11 comprising a clear tent-like structure 23 that is made of clear plastic material of variable stiffness with internally projecting gloved structures 12 and contains instruments 20 needed for the procedure. The device 11 is sterile within and adheres when applied to the skin at opening 13 after removing cover 14 of variable possible dimensions that reveals the opening 13 and a rim of adhesive material of variable possible dimensions 15 that adheres the edges surrounding the opening 13 of the device 11 to the skin. The invention has one or more bracing structures 19 that may be made of a multitude of materials and a multitude of shapes and would be able to support and increase the internal dimensions of the invention and optimize working area as needed. Braces could be mobile and placed at any position in the device 11 as needed for support. Alternatively or additionally the device could be expanded and supported through external loops 22 incorporated into the device at a multitude of locations as needed. The invention may alternatively be manufactured as a unit with a plastic that has an inherent tendency towards keeping the device expanded as would happen with plastic of greater stiffness or one with certain areas of increased stiffness that support expansion of the invention. A bacterial filter 7 allows for sterile air to enter as needed for expansion of the device. A resealable flap 18 and opening 25 could be used in the event of need to add or subtract items from the device in a sterile fashion. A pocket 17 of various possible shapes allows for insertion of an ultrasound probe that may be applied close to the skin and near the site of skin puncture as required to visualize structures such as vascular anatomy and fluid collections that will be engaged during the procedure. A more solid window 24 with greater clarity may be incorporated into the device as needed to improve visualization of the field.

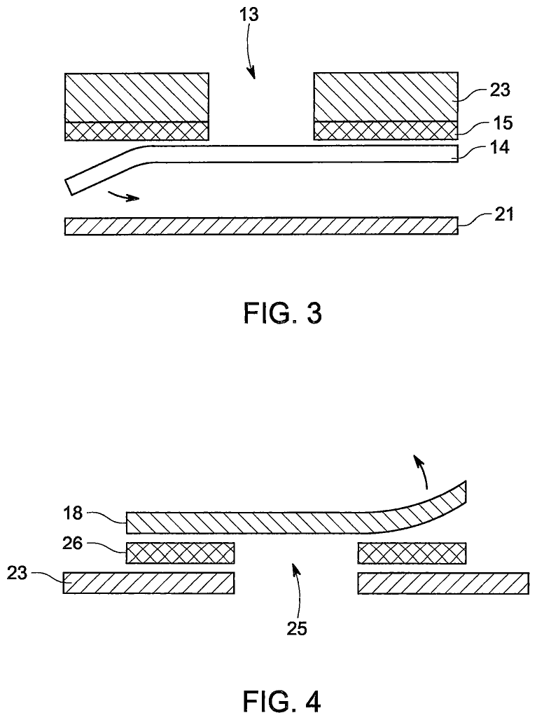

In FIG. 3 an exploded side view of the opening 13 of the device 11 is shown wherein the layers of the tent-like structure 23, adhesive layer 15, and removable protective cover 14 are shown in relation to the skin 21 that it is to be applied to. The protective cover 14 prevents adhesion of the adhesive layer 15 to the skin 21 until the cover 14 is removed.

In FIG. 4 an exploded side view of the resealable flap 18 with opening 25 is seen wherein the resealable flap is adhered to the device 23 by an adhesive material 26 that is present around the perimeter of the flap so as to keep the perimeter of the flap 18 attached to the device 23 and form a complete barrier of the contents of the device 23 to the areas outside the device 23.

The device would be used as follows. When an invasive procedure is required to be performed the area of skin where the procedure will be performed is identified, often assisted by ultrasound localization prior to the procedure, and skin may be marked. The area of the procedure is sterilized with a solution such as chlorhexidine or iodine. The device is removed from a sterile package and the covering over the opening in the device is removed and the adhesive area that secures the device to the skin is applied with care to center the opening over the area where the procedure is to be performed. The sterile contents of the device would be manipulated through hands placed in the gloved projections in the device. The bracing structures will be deployed as needed and the device unfurled and internally expanded to optimize visibility of internal structures with expansion aided by the bacterial filter which allows sterile airflow through the device. The gloved structures facilitate manipulation of instruments, skin puncture, guidewire and catheter placement or whatever is required of the procedure involved. The hands of the operator can be removed from the device during the procedure to achieve other functions such as holding the ultrasound probe or to change operators. An ultrasound probe can be placed in the incorporated pocket provided and the probe can be placed between the device and the skin to visualize structures that may be cannulated or otherwise manipulated during skin puncture. A resealable opening allows for addition and removal of materials. For example if an additional instrument was needed it could be placed through the opening and then the opening could be resealed. After the procedure has been performed the entire device can be removed from the adhesive base that secures the device to the skin. Because the device creates a complete barrier between the sterile invasive work area and the non sterile external work area, sterile attire would be minimized or eliminated thus saving time, materials and reducing exposure to bacterial contamination. Time savings would be achieved as there would be no need to don sterile garb as the device replaces the donning steps. Because the device does not require the usual placement of a sterile ultrasound probe cover, another time consuming step and opportunity for bacterial invasion is avoided. Because the device can cover a reduced area of the patient compared to usual draping procedures, the patient can be better observed for signs of deterioration such as level of alertness and breathing pattern.

1. An internally sterile system for performing percutaneous and other procedures comprising a clear, variably flexible plastic material of variable possible dimensions as required by the procedure to be performed with sterile internal working area and various instruments and devices as needed for performing a procedure contained within the sterile area of the device.

2. The device of claim 1 with one or more gloved internally projecting areas for placing the hands and manipulation of instruments and other devices contained within.

3. The device of claim 1 with an area of attachment of device to skin overlying the area where the procedure is to be performed with an opening of variable possible dimensions and a protective cover of variable possible dimensions over that opening that is peeled away to reveal the opening and a perimeter of an adhesive area of variable possible dimensions under the cover that was peeled away to secure the device in claim 1 to the skin.

4. The device in claim 1 with a pocket of a multitude of possible shapes and locations that allows an ultrasound probe to be placed into the pocket to enable ultrasound interface between the probe, the plastic of the device in claim 1 and the skin and thus facilitate ultrasound guidance of procedures.

5. The device in claim 1 with the expansion of the device as needed supported by mobile bracing material in the interior of the device that may be adjustable in shape and in position along the device as needed to achieve desired visibility and enhance dexterity required for the procedure.

6. The device in claim 1 with integral external loops for use with external bracing or other external support of loops that will be utilized to expand the device as needed.

7. The device in claim 1 with a resealable flap and overlying opening of various possible dimensions in any location on the device that may be opened and all or some needed materials added or subtracted in sterile fashion and the door closed with sterile internal working area preserved using adhesive edges on the flaps of the opening to reseal the opening.

8. The device in claim 1 with a bacterial filter at any location in the device to allow for internal air expansion of the device as needed from its compressed state when the device is first removed from its container.

9. The device in claim 1 with a solid or more flexible window of increased clarity at a multitude of possible locations, sizes and shapes as needed to improve visualization of procedural working areas.
